(19) United States
(12) Patent Application Publication
(10) Pub. No.: US 2011/0076499 A1
(43) Pub. Date: Mar. 31, 2011

(54) Bubble-Containing Solid a-Sulfo Fatty Acid Alkyl Ester Salt and Method for Producing the Same

(75) Inventors: Hiroyuki Masui, Tokyo (JP); Naoki Nakamura, Tokyo (JP); Takao Matsuo, Tokyo (JP)

(73) Assignee: Lion Corporation, Tokyo (JP)

(21) Appl. No.: 12/994,204

(22) PCT Filed: May 25, 2009
(86) PCT No.: PCT/JP2009/059520

§ 371 Date: Nov. 23, 2010

(30) Foreign Application Priority Data
May 23, 2008 (JP) 2008-135586

(51) Int. Cl. C11D 1/28; C07C 53/00; C11D 17/06; B29B 9/12

(52) U.S. Cl. 428/402; 554/85; 264/5

(57) ABSTRACT

A solid a-sulfo fatty acid alkyl ester salt having a bubble volume fraction of 1 to 15% is provided by introducing a gas into a paste of a-sulfo fatty acid alkyl ester salt or adding thereto an expanding agent to incorporate bubbles into the paste of a-sulfo fatty acid alkyl ester salt.

### TECHNICAL FIELD

The present invention relates to a bubble-containing solid α-sulfo fatty acid alkyl ester salt that can be preferably used as a surfactant for constituting the granular detergent composition for clothes, and a production method therefor. In particular, the invention relates to a bubble-containing solid α-sulfo fatty acid alkyl ester salt that can be prevented from adhering to hard surfaces, for example, the inner surface of the apparatus for producing granular detergent compositions, and a production method therefor.

### BACKGROUND ART

The α-sulfo fatty acid alkyl ester salt (α-SF salt) is widely used as a surfactant for producing the granular detergent composition for clothes (see, e.g., WO 2004/111166).

Generally, in order to obtain the granular detergent composition, the spray-dried α-SF salt is mixed with other ingredients, such as a bleaching agent, an inorganic powdery builder and the like. The α-SF salt is commonly transported to a place where to be mixed with other ingredients from the site of spray-drying operation through a conveying pipe installed in the facilities. There produces the problem that the α-SF salt powder may adhere to the elbow of the pipe.

### SUMMARY OF INVENTION

### Technical Problem

Accordingly, an object of the invention is to provide a solid α-sulfo fatty acid alkyl ester salt which can be prevented from adhering to hard surfaces, and a method for producing the above-mentioned solid α-sulfo fatty acid alkyl ester salt.

### Solution to Problem

As a result of intensive studies, the inventors of the present invention have found that the above-mentioned objects can be achieved by a solid α-sulfo fatty acid alkyl ester salt containing bubbles therein with a particular ratio.

Namely, the invention provides a solid α-sulfo fatty acid alkyl ester salt having a bubble volume fraction of 1 to 15%.

The invention also provides a method for producing a solid α-sulfo fatty acid alkyl ester salt having a bubble volume fraction of 1 to 15%, comprising the step of incorporating bubbles into a paste of α-sulfo fatty acid alkyl ester salt.

Further, the invention provides a method for producing a solid α-sulfo fatty acid alkyl ester salt having a bubble volume fraction of 1 to 15%, comprising the step of kneading a solid-state α-sulfo fatty acid alkyl ester salt to incorporate bubbles therein while turning the solid into a paste.

### ADVANTAGEOUS EFFECTS OF INVENTION

The solid product according to the invention can be prevented from adhering to the hard surfaces. Therefore, handling of the product becomes easier in the preparation of detergent compositions, for example, when the product is pneumatically conveyed to the site where to be mixed with other ingredients or the product is pulverized in the presence or absence of other ingredients. In addition, the present invention can provide a solid α-sulfo fatty acid alkyl ester salt having a high degree of whiteness without using any white powder.

### BRIEF DESCRIPTION OF DRAWINGS

FIG. 1 is a section showing the schematic structure of a screw extrusion type granulator used in the examples of the invention.

FIG. 2 is a sectional view showing the schematic structure of a simple acrylic pneumatic conveying system used in the examples of the invention.

### DESCRIPTION OF EMBODIMENTS

The α-sulfo fatty acid alkyl ester salt (α-SF salt) containing bubbles therein according to the invention can be produced by:
introducing a gas into a paste of α-SF salt or adding an expanding agent thereto, or
kneading a solid-state α-SF salt to make the solid into a paste simultaneously with the introduction of a gas therein.

The method of introducing a gas into the paste of α-SF salt or adding an expanding agent thereto according to a first embodiment of the invention will be explained.

### [α-Sulfo Fatty Acid Alkyl Ester Salt in a Paste Form]

### <Raw Materials of α-SF Salt>

Fatty acid esters, preferably fatty acid alkyl esters may be used as the raw materials for preparation of the α-SF salt in the form of a paste. The fatty acid alkyl esters may be used alone or two or more kinds of esters may be used in combination as a mixture. The latter is preferred from the economical viewpoint and in light of the solubility of the α-SF salt in water.

The fatty acids for constituting the fatty acid esters may include saturated or unsaturated straight-chain or branched fatty acids having 8 to 22 carbon atoms, preferably 8 to 18 carbon atoms, more preferably 14 to 18 carbon atoms, for example, capric acid (C10), lauric acid (C12), myristic acid (C14), palmitic acid (C16), stearic acid (C18), and arachidic acid (C20). Advantageously, fatty acid with a lower iodine number can form a solid α-SF salt product with a higher degree of whiteness. More specifically, the iodine number of 0.5 or less is preferable, and 0.2 or less is more preferable. The iodine number may be determined in accordance with JIS K 0070 “Test Method for Acid value, Saponification number, Ester number, Iodine number, Hydroxyl value of Chemical products and Unsaponifiable matter”.

The alcohols used to form the fatty acid esters may include straight-chain or branched monohydric alcohols having 1 to 6 carbon atoms, preferably 1 to 3 carbon atoms, and most preferably one carbon atom.

Preferably, the fatty acid alkyl ester may be prepared from a straight-chain saturated fatty acid with 8 to 18 carbon atoms and a straight-chain alcohol having 1 to 3 carbon atoms. In particular, methyl esters of straight-chain saturated fatty acids having 16 to 18 carbon atoms are preferred.

### <Preparation of α-SF Salt in a Paste Form>

A fatty acid ester as the raw material is first brought into contact with sulfuric anhydride or the like for sulfonation in a thin-film reactor or the like, so that an α-sulfo fatty acid alkyl ester (α-SF) can be obtained. Typically, the molar ratio of the SO₃ to the fatty acid alkyl ester in the reaction may be in the range of 1:1 to 2:1; the reaction time may be 5 to 180 seconds (when the reaction is carried out in the thin-film reactor); and the reaction temperature may be from the melting point to a temperature higher than the melting point by 70° C.

The obtained product is subjected to aging for a given time of period. Generally, the aging step may be carried out by allowing the product to stand at 70 to 100° C. for one to 120 minutes, optionally with stirring.

Then, the resultant α-SF is neutralized with an alkaline chemical, thereby forming the sulfonic acid portion into a salt. Thus, an α-SF salt in a paste form can be obtained. Generally, the neutralization may be carried out at 30 to 140° C. for 10 to 60 minutes. The alkaline chemical used for neutralization includes alkali metal hydroxides, ammonia, amines or the like, preferably alkali metal hydroxides, more preferably sodium hydroxide or potassium hydroxide. In particular, sodium hydroxide is especially preferred. The solid content in the paste obtained after neutralization varies depending upon the concentration of the used alkaline aqueous solution. For example, a paste having a solid content of 60 to 80 mass % can be obtained by the reaction with an alkaline aqueous solution having a concentration of 15 to 50 mass %. Before or after the step of neutralization, the product may be subjected to bleaching using hydrogen peroxide or the like. The solid content herein used can be determined by subtracting the water content and the alcohol content from the total mass.

The α-SF salt in a paste form used in the production method of the invention may be used as it is after obtained in the paste form as previously mentioned, or after removal of the solvent component such as methanol by flash distillation or the like, or after concentrated by evaporating the water content. The apparatus for concentrating the paste is not particularly limited, but a thin-film evaporator is preferred because the concentration can be efficiently carried out even at relatively low temperatures of about 90 to 130° C. The concentration at relatively low temperatures can prevent the α-SF from undergoing hydrolysis. For example, the structure of the thin-film evaporator is such that a cylindrical processing unit with pressure resistance has an inner wall as a heat transfer surface, and a rotary scraper means in the form of a blade (agitating blade) capable of rotating about the shaft is disposed in the processing unit. With respect to the conditions of concentration, the tip peripheral speed of the agitating blade may preferably be 5 to 30 m/s, more preferably 5 to 25 m/s. When the tip peripheral speed is 5 m/s or more, the α-SF salt in a paste form present on the inner wall of the apparatus can be made into a thin film and the liquid can be converted smoothly. At the tip peripheral speed of 30 m/s or less, heat is hardly generated by friction between the wall of the apparatus and the paste of α-SF salt, so that the temperature of the resultant concentrate does not elevate and the mechanical load applied to the thin-film evaporator does not increase. Preferably, the temperature of the heat transfer surface may be 100 to 160° C., and the pressure applied to the inside of the processing unit may be 0.004 MPa to atmospheric pressure. The concentrated paste having a water content reduced up to 5 mass % or less can be efficiently obtained by treating the paste under the above-mentioned conditions of concentration. The water content herein used can be determined using a Karl Fischer moisture titrator (for example, “MKC-210” made by Kyoto Electronics Manufacturing Co., Ltd.).

As the paste of α-SF salt used in the production method of the invention, the α-SF salt obtained in a paste form may be once cooled to temporarily become solid and again turned into a paste for use. For example, the commercially available product of α-SF salt may be turned into a paste form by heating and melting the product or adding a proper amount of water to the product. Alternatively, the paste of α-SF salt prepared by the above-mentioned method is temporarily turned into a solid by cooling and stored in silos, flexible containers or the like. When used, the solid may be reconstituted to a paste form by the kneading step to be described later.

<Step of Incorporating Bubbles into α-SF Salt>

To incorporate bubbles into the α-SF salt in a paste form, the step of physically introducing a gas into the paste of α-SF salt; adding an expanding agent to the paste of α-SF salt to generate bubbles in the paste, or the like can be taken.

Before bubbles are incorporated into the paste, the paste may preferably have a solid content of 95 mass % or more, and more preferably 97 mass % or more. When the solid content is within the above range, the viscosity is high enough to securely retain the bubbles in the paste. The solid content herein used can be determined by subtracting the water content and the alcohol content from the total mass.

The content of the α-SF salt in the solid constituting the paste, which will correspond to the content of α-SF salt in the detergent composition, may determine the content of the surfactant in the detergent composition. Desirably, therefore, the content of the α-SF salt in the solid constituting the paste may be as high as possible, for example, at least 70 mass %, preferably 80 mass % or more, and most preferably 90 mass % or more. The rest in the solid includes unreacted products and by-products generated in the preparation of α-SF salt, so that the content of the α-SF salt in the solid can be increased by eliminating the above-mentioned unreacted products and by-products through the process of extraction, recrystallization or the like.

Prior to the kneading step, the temperature of the paste may be preferably in the range of 50 to 120° C., and more preferably 50 to 110° C. to maintain the high viscosity. The temperature of the paste herein used means the inner temperature of the paste.

The viscosity of the paste before the kneading step is preferably in the range of 100 to 10000 Pa·s, more preferably 500 to 8000 Pa·s in consideration of easy retention of bubbles. The viscosity of the paste can be determined using a HAAKE RheoStress RS75. When the HAAKE RheoStress RS75 is used, the measurement can be carried out under the following conditions, and the viscosity at a shear rate of 0.65 (l/s) can be used as a representative value.

Measuring Mode: CR type γ-Ramp Flow curve
Sh. stress/Sh. rate/Peformation: 0.15-1.2 (l/s)

### Temperature: 60-120° C.

### Time: 180 s.

### Sensor C20/4°

### <Physical Introduction of Gas>

To physically introduce a gas into the α-SF salt, the α-SF salt in a paste form may be mixed with a gas. The kneading step is carried out using a kneader, and the gas can be incorporated into the α-SF salt in the kneader so long as the salt is in a paste form. To incorporate the gas into the paste, the gas may be fed into the paste forcedly by using compressed gas. Alternatively, the air can naturally be incorporated in the paste by leaving the feed hopper of the kneader open.

When the α-SF salt in a paste form is used, it is preferable to feed the α-SF salt in a paste form and a gas into the kneader simultaneously, followed by kneading to mix the gas into the paste forcedly. To force the gas into the paste, a gas compressed by the application of a pressure of 0.1 to 2 MPa in terms of gauge pressure may be preferably used. The feed of the compressed gas may be about 10⁻⁵ to 10⁻² Nm³ per kilogram of the α-SF salt so that the α-SF salt may be amply loaded with the bubbles.

When the α-SF salt in a solid form is used, the solid α-SF salt may be placed into the kneader and then kneaded therein with the feed hopper of the kneader being left open, thereby loading the α-SF salt with a gas as turning the solid α-SF salt into a paste form.

The gas used for forming bubbles is not particularly limited, but it is preferable to use one or more gases selected from the group consisting of the air, nitrogen, carbon dioxide and ammonia, more preferably the air, nitrogen and carbon dioxide, and most preferably the air and nitrogen.

The kneading step may preferably be carried out by the application of a kneading energy of 0.1 to 50 kJ/kg, more preferably 0.3 to 30 kJ/kg, and most preferably 0.5 to 20 kJ/kg, based on the mass of the α-sulfo fatty acid alkyl ester salt in a paste form. The kneading energy can be expressed by the following expressions:

### (1) Batch-Wise Kneading Mode

Kneading energy=(P×t)÷M, where P is the power (kW) necessary for agitation; t is the kneading time (s), and M is the mass (kg) of α-SF salt.

### (2) Continuous Kneading Mode

Kneading energy=P÷v, where P is the power (kW) necessary for agitation; and v is the feed rate, i.e., feeding capability (kg/s) of α-SF salt into the kneader.

In both cases of (1) and (2) as mentioned above, the power (P) necessary for agitation can be determined as follows:

(Case 1) In the case where a running torque meter is set between a motor (i.e., electric motor, internal combustion engine or the like) and the rotary shaft of kneader to determine the running torque and the number of revolutions;

P=T×2π×n÷1000,

where T is the running torque (J), and n is the number of revolutions (rps).
(Case 2) In the case where the load (current) of the motor is read;
2-1: When a single-phase motor is used;

P=E×I×η×pf,

where E is the voltage (V), I is the current (A), η is the motor efficiency (−), and pf is the motor power factor (−).
2-2: When a three-phase motor is used;

P=E×I×√3×η×pf,

where E is the voltage (V), I is the current (A), η is the motor efficiency (−), and pf is the motor power factor (−).

For example, the kneading step may be carried out for 0.1 to 10 minutes when the compressed gas is used to incorporate a gas into the paste forcedly; and 0.3 to 30 minutes when the gas is naturally mixed into the paste by leaving the feed hopper of the kneader open. However, the kneading energy is more influential factor in the incorporation of bubbles, so that it is preferable to change the kneading energy for controlling the bubble volume fraction.

The paste may be heated during the kneading step. The temperature of the paste of α-SF salt may preferably be adjusted to 40 to 95° C., more preferably 45 to 85° C., still more preferably 50 to 80° C., and most preferably 50 to 70° C. during and after the kneading step. When the above-mentioned temperature exceeds 95° C., the bubbles mixed into the α-SF salt may unfavorably condense and disappear in the subsequent cooling and solidifying operation. When the temperature is lower than 40° C., the viscosity of the α-SF salt becomes extremely high, so that the load to the kneader may increase, thereby worsening the productivity.

The kneader that can be used is not particularly limited so long as the kneader is of a continuous type or batch-wise type. The mixers having a blade or the like therein designed to forcibly agitate and mix the contents are included. Examples of the continuous type kneaders include KRC Kneader made by Kurimoto, Ltd.; KEX Extruder made by Kurimoto, Ltd.; SC Processor made by Kurimoto, Ltd.; Extrud-O-Mix, made by Hosokawa Micron Corporation; Twin Taper Single Extruder made by Moriyama Company Ltd.; Feeder Ruder made by Moriyama Company Ltd.; and the like. Examples of the batch type kneaders include batch kneader/pressure kneader made by Kurimoto, Ltd.; Versatile Mixer made by Dalton Co., Ltd.; Open-type mixer made by Moriyama Company Ltd.; Dispersion Mixer made by Moriyama Company Ltd.; Nauta Mixer made by Hosokawa Micron Corporation; LÖDIGE Mixer made by Matsubo Corporation; Ploughshare Mixer made by Pacific Machinery & Engineering Co., Ltd. and the like. In order to smoothly convey the kneaded product to the subsequent step, the kneader of a continuous type is preferred in consideration of the fact that the high viscosity makes the handling of the kneaded product difficult.

In the case where a gas is physically incorporated into the α-SF salt, the bubble volume fraction and the size of bubbles can be controlled by appropriately adjusting the solid content of the α-SF salt paste before the incorporation of bubbles, the pressure upon the introduction of gas, the volume of gas to be introduced, the temperature of the paste in the course of kneading, the kneading time, the kneading manner, the cooling rate after kneading and the like.

### <Addition of Expanding Agent>

The expanding agent may include, for example, ones capable of generating a gas when thermally decomposed, producing a gas when reacting with an acid, and the like.

The expanding agent capable of generating a gas when subjected to thermal decomposition includes the ones that can pyrolyze when the temperature of the kneaded product reaches 60 to 130° C. during the kneading step. More specifically, bicarbonates such as sodium bicarbonate, potassium bicarbonate, ammonium bicarbonate and the like can be employed. In particular, ammonium bicarbonate is preferred because the majority of ammonium bicarbonate is converted to a gas when decomposed.

The expanding agent capable of producing a gas when reacting with an acid includes, for example, sodium bicarbonate and potassium bicarbonate. In particular, sodium bicarbonate is preferred.

As the expanding agent for use in the invention, the ones capable of generating a gas when thermally decomposed are preferred because neither water nor acids are needed in the reaction.

When the expanding agent that can be thermally decomposed to generate a gas is employed, it is preferable that the temperature of the paste be set lower than the temperature where the expanding agent starts to expand when the expanding agent is added to the paste, and the temperature be elevated after the paste is sufficiently kneaded with the expanding agent in order to achieve uniform distribution of bubbles in the paste. Preferably, the temperature of the paste may be elevated to the temperatures between the thermal decomposition temperature of the expanding agent and around 95° C. to efficiently incorporate the bubbles into the paste.

In the case where the expanding agent capable of producing a gas by the reaction with an acid is used, the acid is appropriately added to the paste so that the amount of the acid may be generally within the range of 0.1 to 10 mass % of the paste although the amount of acid varies depending upon the amount and the kind of expanding agent to be added. In this case, the amount of acid to be further added may be decided in consideration of the amount of the α-SF where the sulfonic acid group remains unneutralized and the amount of lower alcohol sulfate present as the by-product in the paste of α-SF salt. Examples of the acid to be added include oxalic acid, citric acid, malic acid, succinic acid, tartaric acid, malonic acid, adipic acid, maleic acid, fumaric acid, polyacrylic acid and the like.

The expanding agent capable of producing a gas by the reaction with an acid will start to react when coming in contact with the acid. In light of this, it is desirable that the expanding agent be speedily mixed into the paste and uniformly distributed therein using the kneader so as to prevent the bubbles from existing unevenly in the paste. By keeping on kneading after completion of the expansion, the bubbles can be distributed more uniformly.

The expanding agent may be added after the α-SF salt is placed into the kneader, or the expanding agent and the α-SF salt may be simultaneously fed into the kneader. In light of the productivity, simultaneous feeding with the α-SF salt is preferred.

The expanding agent may be added preferably in an amount of 5 mass % or less, more preferably 4 mass % or less, based on the solid matter of α-SF-Na. With the expanding agent in an amount of more than 5 mass %, the bubble volume fraction may extremely increase, so that the strength of the resultant solid product will be lowered and the product will get broken by the impact when colliding with the hard surface. Also, the specific surface area of the product will increase, thereby unfavorably increasing the occurrence of adhesion to the hard surface and degrading the degree of whiteness to impair the commercial value of the product. To add the expanding agent in an amount of more than 5 mass % is not recommended from the economical viewpoint because the corresponding effect cannot be obtained if added.

Similarly to the case where the gas is physically introduced, the kneading step may also be carried out by the application of the kneading energy of 0.1 to 50 kJ/kg, more preferably 0.3 to 30 kJ/kg, and most preferably 0.5 to 20 kJ/kg, based on the mass of the α-sulfo fatty acid alkyl ester salt in a paste form when the expanding agent is added.

In the case where the expanding agent is added to the α-SF salt to incorporate bubbles therein, the bubble volume fraction and the size of bubbles can be controlled by appropriately adjusting the solid content of the α-SF salt paste before the incorporation of bubbles, the pressure upon the introduction of gas, the volume of gas to be introduced, the temperature of the paste in the course of kneading, the kneading time, the kneading manner, the cooling rate after kneading, and the like.

The method according to a second embodiment of the invention, i.e., the method of kneading a solid-state α-SF salt to incorporate a gas into the α-SF salt as converting the solid into a paste will be explained.

### <Raw Material of α-SF Salt>

The α-SF salt that can be used as the raw material in the second embodiment is prepared by once cooling the α-SF salt in a paste form as mentioned in the first embodiment to turn the paste into a solid.

<Conversion of Solid-State α-SF Salt into Paste and Incorporation of Gas Therein>

Kneading of the solid-state α-SF salt makes it possible to turn the solid into a paste, and at the same time, to incorporate bubbles into the paste.

A special heat source may not be prepared for the kneading step because the temperature of the solid-state α-SF salt can be easily elevated to turn the solid product into a paste just by converting the kneading energy of the kneader into heat energy, not by applying heat to the solid-state α-SF salt. However, the temperature of the α-SF salt may be elevated, for example, by using a heating medium such as warm water, steam or the like for the jacket of the kneader. It is preferable to turn the solid-state α-SF salt into a paste of 40 to 90° C. in the course of the kneading step, although any method may be employed.

To incorporate a gas into the paste, the gas may be fed into the paste forcedly using compressed gas or the air may be naturally mixed into the paste by leaving the feed hopper of the kneader open in the same manner as mentioned in the first embodiment. The pressure of the compressed gas, the volume of gas to be introduced and the kind of kneader that can be used are the same as those previously described in the first embodiment.

The kneading energy required to knead the solid-state α-SF salt, which energy is used to convert the solid into a paste in the kneader is generally larger than that for kneading the α-SF salt in a paste form initially placed into the kneader. In this case, the kneading energy may preferably be 10 to 500 kJ/kg, more preferably 20 to 400 kJ/kg, and most preferably 35 to 300 kJ/kg. It is particularly preferable to carry out the kneading step with the feed hopper of the kneader being left open. The α-SF salt thus obtained under such conditions can be provided with a satisfactory whiteness effectively without using more energy than needed. The kneading energy can be determined in accordance with the same expressions as described in the previous paragraph with respect to the kneading energy for the α-sulfo fatty acid alkyl ester salt in a paste form.

As a matter of course, in the first embodiment, the bubbles may be incorporated into the paste by introducing a gas in the presence of an expanding agent. Further, in the first or second embodiment, a mixture of the paste of α-SF salt and the solid-state α-SF salt may be used as the starting material, which may be kneaded in the presence or absence of an expanding agent to incorporate the bubbles. These conditions may be used in combination with each other.

The bubble-containing solid-state α-SF salt obtained according to the first and second embodiments is cooled and hardened.

### [Solid α-SF Salt Product]

It can be confirmed whether the obtained solid product of α-SF salt bears bubbles therein by cutting the solid product with a microtome or razor and observing the section by scanning electron microscope (SEM) or the like.

The cooling operation may be carried out by allowing the product to stand at room temperatures or using a drum flaker, belt cooler or the like. Preferably, the α-SF salt may be contained in an amount of 70 mass % or more, and more preferably 80 mass % or more, based on the total mass of the solid product. The content of less than 70 mass % is not appropriate because the resultant detergency will be insufficient when the α-SF salt is blended in a detergent composition.

Prior to the cooling operation, the α-SF salt product may be formed into blocks, briquettes, tablets, noodles, pellets (cylindrical form), flakes, granules and the like if necessary. From a viewpoint of easy handling, pellets, flakes and granules are preferable and pellets are more preferable.

To form the product into noodles, the product previously kneaded in the kneader may be subsequently caused to pass through an extruder (i.e., extruder die) to form noodles. Alternatively, the concentrated product or solid product may be placed into an extruder which is designed to execute both the kneading operation and the extruding operation. The product may be thus formed into noodles through the steps of kneading and extrusion.

The extruders that can be used for obtaining the noodle-shaped product include extrusion granulators described in “Zoryu Handbook (Handbook of Granulation)”, edited by The Association of Powder Process Industry and Engineering, JAPAN (issued by Ohmsha, Ltd.), Chapter 3 (Granulation by extrusion). Those granulators of screw type, roll type, blade type, self-molding type, ram type and the like are usable. The screw type granulators are preferred because high production capability can be obtained. In particular, a preferable structure of the screw type granulator is that at least one paddle, latch and orifice plate are attached and a shearing action can be exerted on the contents. The extrusion linear speed is generally 0.1 to 100 mm/s, preferably 1 to 70 mm/s. When the extrusion speed is within the above-mentioned range, the productivity does not lower and the load applied to the extruder die does not excessively increase. The orifice plates set in the extruder and attached to the end of the extruder may have orifices in any shape, for example, a circle, regular triangle, regular square or the like. The circle orifice is preferable because high strength of the extruder die can be maintained.

The extrusion area of the extruder die is preferably 0.1 to 200 mm²/orifice, more preferably 0.15 to 100 mm²/orifice, still more preferably 0.1 to 3 mm²/orifice, and most preferably 0.15 to 2 mm²/orifice. When the extrusion area is within the above-mentioned range, the productivity does not lower and the load applied to the extruder die does not excessively increase.

The pellets can be obtained by cutting the extruded noodles by means of a cutter disposed at the outlet of the extruder, or crushing the noodles by means of a crusher after the noodles are cooled and hardened. In this case, the crusher having a screen for classification and a rotating blade is preferred. Specifically, Fitzmill (tradename) made by Hosokawa Micron Corporation, New Speed Mill (tradename) made by Okada Seiko Co., Ltd., Comminutor (tradename) made by Fuji Paudal Co., Ltd., Feather Mill (tradename) made by Hosokawa Micron Corporation, Nibbler (tradename) made by Hosokawa Micron Corporation, Roundel Mill (tradename) made by Tokuju Corporation and the like can be used. In addition, a spheronizer having a rotatable disc at the bottom, for example, Marumerizer (made by Dalton Co., Ltd.) can also be used as the crusher. The above-mentioned crushers may be used in combination to perform the step-by-step crushing operation. Further, the pellets of a desired size can be obtained by appropriately setting the crushing conditions. For example, when the crusher equipped with a classification screen and a rotatable blade is used, the pellets with an average diameter of 0.3 to 15 mm and an average length of 0.3 to 100 mm can be obtained by adjusting the opening of the screen for classification and the peripheral speed of the rotatable blade.

To obtain the flakes, the kneaded product may be made into flakes as the product is cooled to about 20 to 40° C. and hardened using a drum flaker, belt cooler or the like.

To obtain the granules, the solid product in the form of flakes, noodles, pellets or the like may be pulverized. Although any granulators may be used without restriction, the granulators having a rotor and a screen therein can be generally used. Preferably used are impact type crushers such as hammer mills, atomizers, pulverizers and the like; cutting/shearing type crushers such as cutter mills, feather mills and the like. In particular, use of the cutter mills, feather mills and speed mills, and the cutting/shearing crushers including granulating and spheronizing equipment such as power mills and the like are advantageous because occurrence of fine dust caused by impact crushing can be reduced. In this case, the cutter blade treated with Stellite (trademark), tungsten carbide or the like may be preferably employed because that kind of blade is less worn away even after a long-time operation. It is preferable that the pulverized product be discharged through a screen with a predetermined opening diameter. To be more specific, Fitzmill (made by Hosokawa Micron Corporation), Speed Mill (made by Okada Sesiko Co., Ltd.), a granulating and spheronizing equipment, Power Mill (made by Dalton Co., Ltd.), Atomizer (made by Fuji Paudal Co., Ltd.), Pulverizer (made by Hosokawa Micron Corporation), Comminutor (made by Fuji Paudal Co., Ltd.) and the like can be used. The screen may be selected with no particular limitation, for example, from woven metal screens, herringbone perforated screens, perforated metal screens and the like. In light of the strength of the screen and the shape of the pulverized product, the perforated metal screens are preferable.

In the course of pulverizing, cool air may be introduced into the pulverizer in order to prevent the pulverized product from being softened by heat generation in the course of the pulverizing operation and attached to the pulverizer. In this case, the temperature of the cool air may be 5 to 30° C., more preferably 5 to 25° C. In addition, the cooling air subjected to dehumidification may be preferably used. The cooling air prepared by diluting the air with nitrogen may be used.

### [Physical Properties of Solid α-SF Salt Product]

### <Bubble Volume Fraction>

The bubble volume fraction herein used is determined in accordance with the following formula (1):

Bubble volume fraction (vol %)=(1−(ρ/ρ₀))×100 (1)

In the formula, ρ indicates the density of a sieve fraction of the α-SF salt containing solid product collected after passing through a first sieve having an opening of 16 mm, but not passing through a second sieve having an opening of 500 μm.

The value ρ₀ indicates the density of a sieve fraction of the α-SF salt containing solid product ground down in an agate mortar or the like, collected after passing through the sieve with an opening of 150 μm.

Both of ρ and ρ₀ are values determined by the air pycnometer. It is possible to use the commercially available air pycnometer, for example, Model 1000, made by Tokyoscience. Co., Ltd. The test sieves as defined in JIS Z8801-1:2006 are used, and the openings are nominal values.

Without wishing to be bound by any theory, it is considered that the solid α-SF salt product could be provided with elasticity by containing the bubbles in a particular proportion, and therefore the solid product tends to less adhere to the solid surface even when coming in contact with the surface or colliding with the surface.

The bubble volume fraction in the solid α-SF salt product according to the invention is 1 to 15%, preferably 3 to 11%. When the bubble volume fraction is less than 1% or more than 15%, the effect of preventing the solid product from adhering to the hard surface may be unsatisfactory. When the bubble volume fraction is less than 1%, the degree of whiteness may also become low. When the bubble volume fraction exceeds 15%, the solid product becomes vulnerable to impact, so that the solid product may cause brittle fracture when running against the inside of the pneumatic conveying pipe and readily adhere to the hard surface in the course of pneumatic conveyance.

### <Whiteness Degree>

The solid α-SF salt product of the invention irregularly reflects light due to fine bubbles distributed in the solid product. Therefore, the whiteness degree of the solid α-SF salt product, even though no white pigment is contained, is nearly equal to that of the case where some white pigment is used. In the solid α-SF salt product according to the invention, the whiteness degree is the lowest when the solid product is in the form of flakes because irregular reflection occurs least. However, even in the case of the flakes, it is preferable that the Hunter whiteness degree (W) in terms of the value (Lab) be 70 or more and the value b* be 20 or less; and more preferably, the Hunter whiteness degree be 72 or more, and the value b* be 15 or less.

### <Size of Bubbles>

The size of the bubbles can be determined by cutting the solid product with a microtome or razor and observing the exposed section and measuring the diameters of the bubbles to calculate the mean value using a scanning electron microscope (SEM).

Preferably, the average diameter of the bubbles may be 500 μm or less, and more preferably 200 μm or less. When the average diameter exceeds 500 μm, the whitening effect is lowered. There is no particular lower limit of the average diameter, but generally the lower limit is 0.1 μm or more. When the average diameter is less than 0.1 μm, special steps for reducing the diameter of bubbles are required, which may unfavorably decrease the productivity.

### [Dimensions of Solid α-SF Salt Product]

When the solid product of the invention is in the form of flakes, the average of the average two-axis diameter may be 1.0 to 30.0 mm, the average thickness may be 0.5 to 5.0 mm, and the average elongation may be 1.0 to 59.0. The terms “average of the average two-axis diameter”, “average thickness” and “average elongation” herein used are respectively defined as follows:

### <Average of the Average Two-Axis Diameter>

The average (R) of the average two-axis diameter herein used is the average of the average diameter (r) calculated from the two axis measurements in the mass base distribution of the corresponding samples. Here, the average two-axis diameter (r) can be determined from the shorter diameter (b) and the longer diameter (l) in accordance with the following formula (1):

Average two-axis diameter (r)=(shorter diameter (b)+longer diameter (l))/2 (1)

### (Method for Measuring Shorter Diameter (b) and Longer Diameter (l))

### Measuring Method 1:

When the values of the shorter diameter (b) and the longer diameter (l) are both relatively large (about 10 to 250 mm), vernier calipers may be used for the measurement. The longer diameter (l) is the length of the longest portion of the solid product. After 100 samples of the solid product are collected by random sampling, the longest portion of each sample is measured and then the average may be obtained as the longer diameter (l). The shorter diameter (b), which has the maximum length perpendicular to the above-mentioned longer diameter can be measured in the same manner as in the case of the longer diameter (l).

### Measuring Method 2:

When the values of the shorter diameter (b) and the longer diameter (l) are both relatively small (about 0.1 to 10 mm), 1500 to 2000 pictures of the sample are taken from directions at right angles with respect to the vertical direction of the sample, using a digital image processing particle size distribution analysis system (e.g., CAMSIZER made by HORIBA, Ltd.). The distance between two tangents of an image (feret diameter) is measured from 64 angles and the longest feret diameter thus obtained is regarded as the longer diameter. A diameter that is perpendicular to the longer diameter and has the maximum length is regarded as the shorter diameter (b), which can be measured in the same manner as in the case of the longer diameter (l).

### <Average Thickness>

The average thickness (T) is a mean of the thickness (t) in the mass base distribution of the corresponding samples. Here, the thickness (t) is regarded as the maximum distance between parallel planes that are parallel to a referential horizontal plane and tangential to the surfaces of a flake, as defined by Heywood, the referential horizontal plane being chosen as a plane of greatest stability on which the flake can stand still. The thickness (t) can be measured with vernier calipers.

### <Average Elongation>

The average elongation (D) is a mean of the elongation (d) in the mass base distribution of the corresponding samples. Here, the elongation (d) is determined from the above-mentioned shorter diameter (b) and longer diameter (l) in accordance with the following formula (2):

Elongation (d)=Longer diameter (l)/Shorter diameter (b) (2)

When the product is in the form of a granule, the average diameter may be preferably 0.2 to 1.2 mm, and more preferably 0.3 to 1.0 mm.

When the product is in a pellet form, the average diameter may be preferably 0.3 to 15 mm, more preferably 0.3 to 2 mm, and further more preferably 0.5 to 1.5 mm. The average length may be preferably 0.3 to 100 mm, more preferably 0.3 to 10 mm, and still more preferably 0.5 to 5 mm.

When the solid product of such dimensions as mentioned above has a bubble volume fraction within the range previously defined, not only the adhesion to the hard surface can be prevented, but also it is possible to obtain a solid α-SF salt product of which the whiteness degree (to be obtained without any white pigment) is almost the same as that obtained in the case where some white pigment is used.

Each dimension can be measured using vernier calipers regardless of the shape of the solid product.

By using the solid α-SF salt product of the invention in combination with other ingredients if desired, for example, other surfactants than the α-SF salt, an inorganic or organic builder, an alkaline chemical, an enzyme, a recontamination inhibitor, a perfume, a fluorescent brightener, a bleaching agent and the like, powder detergent compositions can be manufactured.

### EXAMPLES

### Preparation Example 1

### Preparation of α-SF Salt in a Paste Form

In a 1-kL reaction vessel equipped with a stirrer, 330 kg of a fatty acid methyl ester mixture (prepared by previously mixing methyl palmitate (“PASTELL M-16”, made by Lion Corporation) and methyl stearate (“PASTELL M-180” made by Lion Corporation) at a ratio by mass of 9:1) was introduced, and then anhydrous sodium sulfate was added with stirring as the coloring inhibitor in an amount of 5 mass % with respect to the above-mentioned fatty acid methyl ester mixture. While keeping on stirring, 110 kg (equivalent to 1.2 moles to one mole of the fatty acid methyl ester mixture) of SO₃ gas (sulfonating gas) which was diluted to 4 vol % with nitrogen gas was blown into the mixture with bubbling, at a constant rate over a period of 3 hours at the reaction temperature of 80° C. The reaction mixture was then subjected to aging at 80° C. for 30 minutes.

Then, the reaction mixture was esterified by the addition of 14 kg of methanol as the lower alcohol. The esterification reaction was carried out at 80° C. The reaction mixture was then subjected to aging for 30 minutes.

Next, the esterified product extracted from the reaction vessel was continuously neutralized by the addition of an equivalent amount of sodium hydroxide aqueous solution, using a line mixer.

Subsequently, the above-mentioned neutralized product was fed into a bleaching agent mixing line, where a 35% hydrogen peroxide solution was added in an amount of 1% with respect to the anionic surfactant concentration (i.e., the total of concentrations of the α-sulfo fatty acid methyl ester sodium salt and the α-sulfo fatty acid disodium salt (di-Na salt)) in terms of the active ingredient. The bleaching step was carried out at 80° C., so that an α-SF salt (α-SF-Na) was obtained in a paste form.

The color tone of the obtained paste was found to be 30. To determine the color tone, the obtained paste was diluted with ethanol so that the anionic surfactant concentration (i.e., the total of concentrations of the α-sulfo fatty acid methyl ester sodium salt and the α-sulfo fatty acid disodium salt (di-Na salt)) reached 5 mass %. The color tone of the resultant solution was measured using a Klett photometric colorimeter with a blue filter No. 42 (optical path length: 40 mm).

### [Concentration of α-SF-Na Paste]

At a feed rate of 35 kg/hour the above-mentioned α-SF-Na paste was fed into a vacuum thin-film evaporator EXEVA (trade name, made by Shinko-Pantec Co., Ltd., having a heat-transfer area of 0.5 m², a cylindrical processing unit with an inner diameter of 205 mm, with a clearance between the scraper blade tips and the heat-transfer wall being 3 mm), which was rotated at 1060 rpm, with the blade tips being rotated at a velocity of about 11 m/sec. The paste was concentrated under the conditions that the inner wall heating temperature (i.e., the temperature of the heat-transfer wall) was 135° C. and the degree of vacuum (i.e., the pressure applied to the inside of the processing unit) was 0.007 to 0.014 MPa.

The temperature of the concentrated paste was 115° C. and the water content thereof was 2.5%. The water content was determined using a Karl Fischer moisture titrator (“MKC-210” made by Kyoto Electronics Manufacturing Co., Ltd., in accordance with Method 2, at a stirring rate of 4). The sample amount was about 0.05 g.

The composition of the concentrated paste was determined in the following manners. The results are shown below.

| | |
|---|---|
| α-SF-Na | 85.3 mass % |
| water content | 2.5 mass % |
| methyl sulfate | 6.1 mass % |
| sodium sulfate | 2.5 mass % |
| α-sulfo fatty acid disodium salt | 3.6 mass % |
| methanol | trace |
| unreacted methyl ester | trace |
| others | trace |
| | 100 mass % |

[Anionic Surfactant Concentration (Total of Concentrations of α-sulfo Fatty Acid Methyl Ester Sodium Salt and α-sulfo Fatty Acid Disodium Salt (di-Na Salt))]

0.3 g of the paste was accurately weighed into a 200-mL volumetric flask. Deionized water (distilled water) was added up to the calibration mark to dissolve the paste therein under application of ultrasonic wave. The resultant solution was cooled to about 25° C. and a portion (5 mL) thereof was transferred to a vessel for titration using a whole pipette. To this solution, 25 mL of methylene blue indicator and 15 mL of chloroform were added, and 5 mL of benzethonium chloride solution with a concentration of 0.004 mol/L was further added. Then, the solution was titrated with a 0.002 mol/L sodium alkylbenzenesulfonate solution. Each time the sodium alkylbenzenesulfonate solution was dropwise added, the vessel was stoppered and vigorously shaken and thereafter allowed to stand. The end point was determined when the two phases showed the same color tone against the background of a white plate. A blank experiment was carried out in the same manner as in the above except that no paste was employed. The anionic surfactant concentration was calculated from the difference in titer.

[Ratio of di-Na Salt in Anionic Surfactants]

0.02 g, 0.05 g and 0.1 g of a standard product of di-Na salt were accurately weighed, and separately placed into a 200-mL volumetric flask. Approximately 50 mL of water and 50 mL of ethanol were added to dissolve the salt therein under application of ultrasonic wave. The resultant solution was cooled to about 25° C. and methanol was added up to the calibration mark precisely. The standard solutions were thus prepared.

After about 2 mL of the standard solution was filtered through a filter of 0.45-μm Chromatodisc, and then subjected to high performance liquid chromatography under the conditions as shown below, thereby obtaining a calibration curve from the peak area.

### (Measurement Conditions of High Performance Liquid Chromatography)

Instrument: LC-6A (made by Shimadzu Corporation.)

Column: Nucleosil 5SB (made by GL Sciences Inc.)

Column temperature: 40° C.

Detector: Differential refractometer detector RID-6A (made by Shimadzu Corporation.)

Mobile phase: 0.7% solution of sodium perchlorate in a mixture of H₂O and CH₃OH (mixing ratio by volume of 1:4)

Flow rate: 1.0 mL/min

Injected amount: 100 μL

Next, 1.5 g of the paste was accurately weighed into a 200-mL volumetric flask. Approximately 50 mL of water and 50 mL of ethanol were added to dissolve the paste therein under application of ultrasonic wave. The resultant solution was cooled to about 25° C. and methanol was added up to the calibration mark precisely, so that a sample solution was prepared.

After about 2 mL of the sample solution was filtered through a filter of 0.45-μm Chromatodisc, and then subjected to high performance liquid chromatography under the same conditions as given above. Using the calibration curves obtained from the above, the concentration of di-Na salt in the sample solution was determined.

From the concentration of di-Na salt thus obtained and the total of the concentrations of anionic surfactants previously obtained, the amount ratio (in terms of percent by mass) of di-Na salt in the anionic surfactants was determined by calculation. Also, the ratio (in terms of percent by mass) of the α-sulfo fatty acid methyl ester sodium salt and that of the α-sulfo fatty acid disodium salt (di-Na salt) in the α-SF-Na paste were individually calculated.

### [Concentrations of Sodium Sulfate and Methyl Sulfate (Mass %)]

0.02 g, 0.04 g, 0.1 g and 0.2 g of each standard product of methyl sulfate and sodium sulfate were accurately weighed, and separately placed into a 200-mL volumetric flask. Deionized water (distilled water) was added up to the calibration mark to dissolve the product therein under application of ultrasonic wave. Each of the resultant solutions was cooled to about 25° C. The standard solutions were thus prepared. After about 2 mL of the standard solution was filtered through a filter of 0.45-μm Chromatodisc, and then subjected to ion chromatography under the conditions as shown below, thereby obtaining a calibration curve from the peak area of the methyl sulfate standard solution and the sodium sulfate standard solution.

### (Measurement Conditions of Ion Chromatography)

Instrument: DX-500 (made by Nippon Dionex K.K.)

Detector: Conductivity detector CD-20 (made by Nippon Dionex K.K.)

Pump: IP-25 (made by Nippon Dionex K.K.)

Oven: LC-25 (made by Nippon Dionex K.K.)

Integrator: C-R6A (made by Shimadzu Corporation.)

Separation column: AS-12A (made by Nippon Dionex K.K.)

Guard column: AG-12A (made by Nippon Dionex K.K.)

Eluting solution: 5 vol. % acetonitrile aqueous solution of Na₂CO₃ (2.5 mM) and NaOH (2.5 mM)

Flow rate of eluting solution: 1.3 mL/min

Regeneration solution: pure water

Column temperature: 30° C.

Loop capacity: 25 μL

Next, 0.3 g of the paste was accurately weighed into a 200-mL volumetric flask. Deionized water (distilled water) was added up to the calibration mark to dissolve the paste therein under application of ultrasonic wave. The resultant solution was cooled to about 25° C., which was used as a sample solution.

After about 2 mL of the sample solution was filtered through a filter of 0.45-μm Chromatodisc, and then subjected to ion chromatography under the same conditions as given above. Using the calibration curves obtained from the above, the concentration of methyl sulfate and that of sodium sulfate in the sample solution were determined and calculated in terms of percent by mass.

### [Concentrations of Methanol and Unreacted Methyl Ester (Mass %)]

According to the conventional analysis by gas chromatography, the concentration of methanol and the concentration of unreacted methyl ester were separately calculated from the ratio of the peak area in the sample product to that of the standard product.

### Preparation Example 2

### Preparation of α-SF Salt in a Paste Form

A paste of α-SF salt was prepared in the same manner as in Preparation Example 1 except that the mixing ratio by mass of methyl palmitate (“PASTELL M-16”, made by Lion Corporation) to methyl stearate (“PASTELL M-180” made by Lion Corporation) in the fatty acid methyl ester mixture as the raw material was changed to 8:2. The color tone of the obtained paste was 40.

### [Concentration of α-SF-Na Paste]

The α-SF-Na paste was concentrated in the same manner as in Preparation Example 1. The temperature of the concentrated paste was 115° C. and the water content thereof was 2.0%.

The composition of the concentrated paste is shown below.

| | |
|---|---|
| α-SF-Na | 85.1 mass % |
| water content | 2.0 mass % |
| methyl sulfate | 6.3 mass % |
| sodium sulfate | 2.7 mass % |
| α-sulfo fatty acid disodium salt | 3.9 mass % |
| methanol | trace |
| unreacted methyl ester | trace |
| others | trace |
| | 100 mass % |

### Example 1

Along with the compressed air (25° C.) at a gauge pressure of 0.5 MPa, the whole amount of the α-SF-Na paste obtained in Preparation Example 1 was continuously fed into a feed hopper of a continuous kneader (KRC kneader, model S-2, made by Kurimoto, Ltd.) so that the ratio of the compressed air to the paste was 10⁻³ Nm³/kg. Upon feeding, the temperature of the paste was 110° C. and the feed rate was 90 kg/h. Water of 20° C. (at the inlet) was caused to pass through the jacket. When the feeding of the entire amount of paste was completed, the charging of the compressed air was stopped. The α-SF-Na paste was kneaded together with the compressed air, with the main shaft of the kneader being rotated at 100 rpm. The kneading energy (kJ/kg) is shown in Table 1. The kneading energies in Examples 1 to 6 (shown in Tables 1 and 2) were determined by measuring the current and the voltage supplied to the motor of the kneader using an ammeter and a voltmeter, followed by calculation in accordance with the formula described in the previous paragraph, corresponding to the continuous kneading mode (2) where the three-phase motor is used (2-2) in the Case 2).

After completion of the kneading, the kneaded product was discharged from the outlet of the kneader. The temperature of the kneaded product thus obtained was 90° C. At a feed rate of 222 kg/h the kneaded product was continuously fed to a double belt cooler (NR3-Lo. Cooler, made by Nippon Belting Co., Ltd.) where the clearance between the feeding pulleys was adjusted to 2 mm. The cooling operation was conducted under the conditions that the travelling speed of the belt was 6 m/s, the flow rate of cooling water on the upper side was 1500 L/h (by flowing water on the rear surface of the belt in a counterflow manner), the flow rate of cooling water on the lower side was 1800 L/h (by spraying the water onto the rear surface of the belt) and the temperature of cooling water supplied was 20° C. A surfactant-containing sheet thus discharged from the cooling belt was then crushed at 200 rpm using the accessory crusher located adjacent to the discharging pulley of the belt cooler, thereby obtaining a solid α-SF-Na product in the form of flakes of which temperature was 23° C.

TABLE A
(For reference) Specifications of Double Belt Cooler (NR3-Lo. Cooler)
made by Nippon Belting Co., Ltd.

| | |
|---|---|
| Distance between pulley cores | 3270/3630 |
| (upper pulley/lower pulley) [mm] | |
| Dimensions of belt [mm] | Upper: 8440 × 650 × 0.8 |
| (L × W × T) | Lower: 9640 × 620 × 0.8 |
| Belt speed [m/min.] | 0.4-18 |
| Effective cooling length [m] | 3.1 |
| Effective cooling width [m] | 0.47 |

### Examples 2 to 4

The solid α-SF-Na product in the form of flakes was obtained in the same manner as in Example 1 except that the feed rate of the α-SF-Na paste fed into the continuous kneader was changed to control the temperature of the kneaded product and the kneading energy.

### Comparative Example 1

At a feed rate of 222 kg/h the concentrated α-SF-Na paste obtained in Preparation Example 1 was continuously fed to a double belt cooler (NR3-Lo. Cooler, made by Nippon Belting Co., Ltd.) where the clearance between the feeding pulleys was adjusted to 2 mm. The cooling operation was conducted under the conditions that the travelling speed of the belt was 6 m/s, the flow rate of cooling water on the upper side was 1500 L/h (by flowing water on the rear surface of the belt in a counterflow manner), the flow rate of cooling water on the lower side was 1800 L/h (by spraying the water onto the rear surface of the belt) and the temperature of cooling water supplied was 20° C. A surfactant-containing sheet thus discharged from the cooling belt was then crushed at 200 rpm using the accessory crusher located adjacent to the discharging pulley of the belt cooler, thereby obtaining a solid α-SF-Na product in the form of flakes of which temperature was 25° C.

### Comparative Example 2

The concentrated α-SF-Na paste obtained in Preparation Example 1 was sandwiched between two stainless-steel sheets (with a thickness of 1 mm, 10 cm×10 cm) so that the thickness of the paste reached 6 mm and cooled in an atmosphere of 20° C. until the paste temperature reached 25° C. The plate-shaped product obtained after cooling was crushed using a crusher, New Speed Mill ND-10 (made by Okada Sesiko Co., Ltd.) equipped with a screen having an opening of 30 mm, with the number of revolutions being set to 840 rpm, thereby obtaining a solid α-SF-Na product in the form of flakes.

### Example 5

Along with the compressed air (25° C.) at a gauge pressure of 0.5 MPa, the α-SF-Na paste obtained in Preparation Example 1 was continuously fed into a feed hopper of a continuous kneader (KRC kneader, model S-2, made by Kurimoto, Ltd.) so that the ratio of the compressed air to the paste was 10⁻³ Nm³/kg. Upon feeding, the temperature of the paste was 110° C. and the feed rate was 20 kg/h. Water of 20° C. (at the inlet) was caused to pass through the jacket. When the feeding of the entire amount of paste was completed, the charging of the compressed air was stopped. The α-SF-Na paste was kneaded together with the compressed air, with the main shaft of the kneader being rotated at 100 rpm. The temperature of the kneaded product thus obtained was 70° C.

After completion of the kneading, the kneaded product was sandwiched between two stainless-steel sheets (with a thickness of 1 mm, 10 cm×10 cm) so that the thickness of the kneaded product reached 6 mm, and cooled in an atmosphere of 20° C. until the temperature of the product reached 25° C. Thus, a solid α-SF-Na product was obtained in the form of flakes.

### Example 6

The α-SF-Na paste obtained in Preparation Example 1 was continuously fed into a feed hopper of a continuous kneader (KRC kneader, model S-2, made by Kurimoto, Ltd.), with the feed hopper being left open. Upon feeding, the temperature of the paste was 110° C. and the feed rate was 20 kg/h. Water of 20° C. (at the inlet) was caused to pass through the jacket. The α-SF-Na paste was kneaded together with the compressed air with the main shaft of the kneader being rotated at 100 rpm by the application of energy of 20 kJ/kg. The temperature of the kneaded product thus obtained was 70° C.

At a feed rate of 222 kg/h the kneaded product was continuously fed to a double belt cooler (NR3-Lo. Cooler, made by Nippon Belting Co., Ltd.) where the clearance between the feeding pulleys was adjusted to 2 mm. The cooling operation was conducted under the conditions that the travelling speed of the belt was 6 m/s, the flow rate of cooling water on the upper side was 1500 L/h (by flowing water on the rear surface of the belt in a counterflow manner), the flow rate of cooling water on the lower side was 1800 L/h (by spraying the water onto the rear surface of the belt) and the temperature of cooling water supplied was 20° C. A surfactant-containing sheet thus discharged from the cooling belt was then crushed at 200 rpm using the accessory crusher located adjacent to the discharging pulley of the belt cooler, thereby obtaining a solid α-SF-Na product in the form of flakes of which temperature was 22° C.

### Example 7

Six kilogram of the concentrated α-SF-Na paste obtained in Preparation Example 1 was fed into a vertical kneader (Versatile Mixer 25AM-RR, made by Dalton Co., Ltd., hook type mixer). Upon feeding, the temperature of the paste was 110° C. Water of 20° C. (at the inlet) was caused to pass through the jacket.

Subsequently, 0.2 kg of ammonium bicarbonate (made by Kanto Chemical Co., Inc., CICA first grade reagent) was added as the expanding agent, and the kneading operation was started. The kneading was continued for 20 minutes, with the kneader being caused to rotate on its axis at 40 rpm and revolve at 26 rpm, so that the kneaded product of 70° C. was obtained.

The kneading energy (kJ/kg) in Example 7, which is shown in Table 2 was obtained by measuring the current and the voltage supplied to the motor of the kneader using an ammeter and a voltmeter, followed by calculation in accordance with the formula described in the previous paragraph, corresponding to the batch-wise kneading mode (1) where the three-phase motor is used (2-2) in the Case 2.

After completion of the kneading operation, the kneaded product was sandwiched between two stainless-steel sheets (with a thickness of 1 mm, 10 cm×10 cm) so that the thickness of the kneaded product reached 6 mm and cooled in an atmosphere of 20° C. until and the product temperature reached 25° C. The plate-shaped product obtained after cooling was crushed using a crusher, New Speed Mill ND-10 (made by Okada Sesiko Co., Ltd.) equipped with a screen having an opening of 30 mm, with the number of revolutions being set to 840 rpm, thereby obtaining a solid α-SF-Na product in the form of flakes.

### Example 8

Along with dehumidified cool air of 15° C. (dew point: −5° C., airflow volume: 6 Nm³/min), the solid α-SF-Na product in the form of flakes obtained in Example 1 was introduced into a pulverizer, Fitzmill DKA-3 made by Hosokawa Micron Corporation, having two-stage screens arranged in series (the opening diameter of the first-stage screen: 8 mm, the opening diameter of the second-stage screen: 3.5 mm, the number of revolutions of the first-stage blade: 4700 rpm, and the number of revolutions of the second-stage blade: 2820 rpm). The flakes were pulverized at a throughput speed of 200 kg/hr, so that the solid α-SF-Na product was obtained in the form of granules with an average particle diameter of 500 μm.

### (Determination of Average Particle Diameter)

Classification was conducted using nine sieves with the respective openings of 1680, 1410, 1190, 1000, 710, 500, 350, 250 and 149 μm, and a saucer. For the classification, above the saucer the sieves were arranged above top of each other in ascending order of opening, and 100 g of the sample of spray-dried granules was placed into the uppermost sieve of 1680 μm and the lid was closed. The classifier was set in the Ro-Tap sieve shaker (made by Iida-seisakusho Japan Corporation, 156 tappings/min, 290 rollings/min) and oscillations were applied to the classifier for 10 minutes under the circumstances of 25° C. and 40% RH. After that, the sample granules remaining on each sieve and the saucer were separately collected.

By repeating the above-mentioned procedures, a sample classified according to the particle diameter, i.e., 1410 to 1680 μm (1410 μm on), 1190 to 1410 μm (1190 μm on), 1000 to 1190 μm (1000 μm on), 1000 to 710 μm (710 μm on), 500 to 710 μm (500 μm on), 350 to 500 μm (350 μm on), 250 to 350 μm (250 μm on), 149 to 250 μm (149 μm on), and the saucer to 149 μm (149 μm pass) was obtained, and then the mass frequency percent (%) was calculated.

Next, the opening of the sieve which first showed a mass frequency percent of 50% or more was supposed to be a (μm); the opening of the sieve next higher than the sieve having an opening of a (μm) was supposed to be b (μm); the cumulative finer mass frequency percent from the saucer to the sieve having an opening of a (μm) was supposed to be c (%); and the mass frequency percent on the sieve having an opening of a (μm) was supposed to be d (%). Then, the average particle diameter (by mass-frequency percent of 50%) was determined according to the following formula:

Average particle diameter (by mass-frequency percent of 50%)=10^{(50-(c-d/log b-log a)×log b))/(d/(log b-log a))}

### Comparative Example 3

The solid α-SF-Na product in the form of granules with an average particle diameter of 500 μm was obtained in the same manner as in Example 8 except that the solid α-SF-Na product in the form of flakes obtained in Comparative Example 1 was used.

### Examples 9 to 24

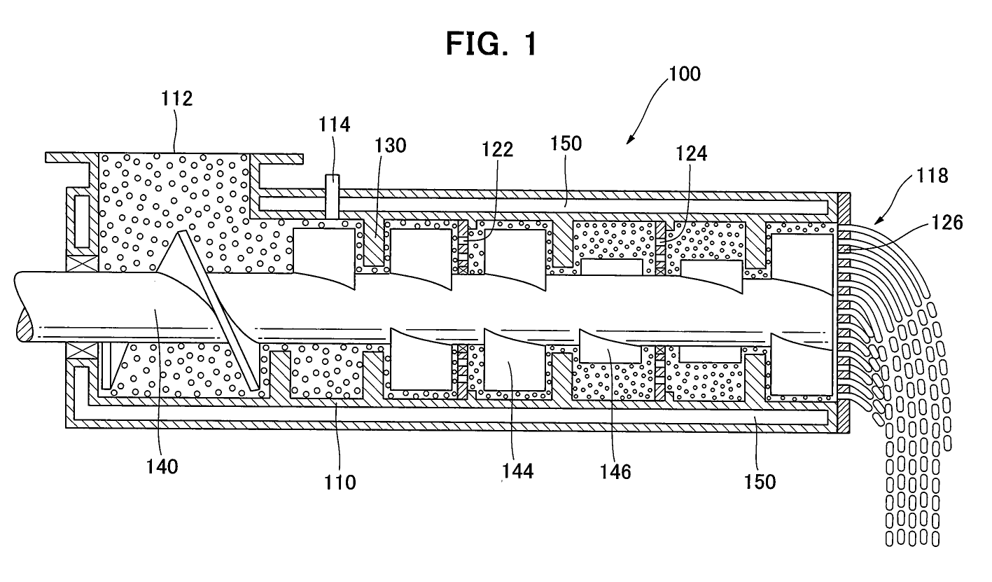

As a screw extrusion granulator 100 used in the corresponding Examples, Extrud-O-Mix Model EM-6 (made by Hosokawa Micron Corporation, with the number of revolutions of the main shaft being 70 rpm), was employed. With reference to FIG. 1, a screw extrusion granulator 100 is provided with a cylindrical casing 110, a feed hopper 112 disposed upstream on the upper side of the casing 110, a feed opening for binder 114 disposed on the upper side of the casing 110 downstream with respect to the feed hopper 112, and an outlet of product 118 disposed at the downstream end of the casing 110. A rotatable screw shaft 140 is positioned in the casing 110. The screw shaft 140 is rotated by the rotation of a driving mechanism such as a motor (not shown) or the like. In the casing 110 of the screw extrusion granulator, there are disposed a latch 130 fixed downstream from the binder feed opening 114 (not used in the invention), a first paddle 144 disposed downstream from the latch 130, a second paddle 146 disposed downstream from the first paddle 144, a first orifice plate 122 located on the upstream side and a second orifice plate 124 located on the downstream side. The first orifice plate 122 disposed closer to the feed hopper 112 has orifices with a diameter of 6 mm. The orifices of the second orifice plate 124 have a diameter of 4.5 mm. Further, a third orifice plate 126 is positioned at the product outlet 118. The shape, diameter and area of the orifice on the third orifice plate 126 used in each of the Examples are shown in Tables 3 to 5. The first paddle 144 on the upstream side and the second paddle 146 on the downstream side are fixed to the casing 110. By the rotation of the screw shaft 140, the first paddle 144 and the second paddle 146 are also rotated along with the rotation of the screw shaft 140. The casing 110 of the screw extrusion granulator is enclosed by a jacket 150. Water is caused to pass through the jacket 150 so that the temperature of water is 30° C. at the inlet.

The flakes of solid α-SF-Na product obtained in Comparative Example 1 were continuously fed into the feed hopper 112 of the screw extrusion granulator 100 at a feed rate of 20 to 200 kg/h, with the feed hopper 112 being left open. Upon feeding, the temperature of the flakes was 25° C. By rotating the screw shaft 140 to revolve the first paddle 144 and the second paddle 146 at 70 to 160 rpm, the solid α-SF-Na product was kneaded and extruded from the orifices of the third orifice plate 126, thereby obtaining the solid α-SF-Na product in the form of noodles.

The kneading energies (kJ/kg) in Examples 9 to 24, which are shown in Tables 3 to 5 were obtained by measuring the current and the voltage supplied to the motor of the kneader using an ammeter and a voltmeter, followed by calculation in accordance with the formula described in the previous paragraph, corresponding to the continuous kneading mode (2) where the three-phase motor is used (2-2) in the Case 2.

After the noodles were allowed to stand at room temperature and cooled to 30° C., the noodle-shaped product was fed into a pulverizer, Speed Mill ND-10, made by Okada Sesiko Co., Ltd., equipped with a screen having a 2-mm opening (or a screen having a 4-mm opening to pulverize the solid α-SF-Na product in the form of noodles obtained by passing through the third orifice plate with an orifice diameter of 1.5 mm or more) at a feed rate of 1 kg/min and pulverized, with the blade being rotated at 840 rpm, thereby obtaining a solid α-SF-Na product in the form of pellets.

### Example 25

The solid α-SF-Na product in the form of flakes obtained in Comparative Example 1 was continuously fed into a feed hopper of a continuous kneader (KRC kneader, model S-4, made by Kurimoto, Ltd.) at a feed rate of 225 kg/h and kneaded under the conditions that the number of revolutions of the main shaft was 130 rpm and the kneading energy was 30 kJ/kg (provided that the temperature of the flakes was 30° C. and the jacket temperature was 80° C.). Then, the kneaded product of 55° C. was obtained.

The kneaded product thus obtained was then continuously fed into an extruder (Pelleter Double EXDJF-100, made by Dalton Co., Ltd., having a die with an orifice diameter of 0.8 mm) at a feed rate of 225 kg/h and subjected to extrusion. The extrusion conditions were that the number of revolutions of the main shaft was 72 rpm, the kneading energy was 10 kJ/kg, and the jacket temperature was 25° C. The temperature of the thus extruded product was 60° C.

Subsequently, the extruded product thus obtained was allowed to stand on a 2-mm mesh so that the thickness of the product reached 60 mm, and then cooled to 30° C. by passing air (15° C. and 30% RH) at a superficial velocity of 1.5 m/s.

After completion of the cooling operation, the extruded product was continuously fed into a crusher (Nibbler NBS300/450 made by Hosokawa Micron Corporation, equipped with a screen having an orifice diameter of 14 mm) at a feed rate of 225 kg/h, and crushed at 75 rpm, so that the crushed product was obtained.

Finally, the crushed product thus obtained and A type zeolite were fed into a pulverizer, Fitzmill DKA-6 made by Hosokawa Micron Corporation, at a feed rate of 190 kg/h and 10 kg/h, respectively, so that a solid α-SF-Na product in the form of pellets was obtained. The pulverizing operation was conducted under the conditions that the screen opening (diameter) was 3 mm and the number of revolutions was 2350 rpm.

### Example 26

The concentrated α-SF-Na paste obtained in Preparation Example 2 was formed into flakes in the same manner as in Comparative Example 1. The solid α-SF-Na product in the form of flakes thus obtained was formed into pellets in the same manner as in Example 25 except that the kneading energy of the continuous kneader was changed to 11 kJ/kg and the kneading energy of the extruder was changed to 9 kJ/kg.

### Example 27

A solid α-SF-Na product in the form of granules (with an average particle diameter of 510 μm) was obtained in the same manner as in Example 26 except that the orifice diameter of the extruder was changed to 5 mm and the kneading energy of the extruder was changed to 7 kJ/kg, and the screen opening (diameter) and the number of revolutions of the pulverizer were respectively change to 2 mm and 3400 rpm.

### Example 28

A solid α-SF-Na product in the form of pellets was obtained in the same manner as in Example 26 except that the orifice diameter of the extruder was changed to 10 mm and the kneading energy of the extruder was changed to 7 kJ/kg, and the pulverizing operation was not conducted after crushing operation.

### [Evaluations of Physical Properties of Solid α-SF-Na Products]

The bubble volume fraction, adhesion properties, whiteness degree, and the shape and thickness of the solid α-SF-Na products obtained in Examples and Comparative Examples were evaluated in accordance with the following methods.

### (1) Method for Determining the Bubble Volume Fraction

### (1)-1: Preparation of Sample for Measuring the Density (ρ₀)

Each of the solid α-SF-Na products obtained was ground down in an agate mortar, and subjected to screening with a sieve having an opening of 150 μm. The fraction passing through the above-mentioned sieve was used as a sample for measuring the density (ρ₀).

### (1)-2: Preparation of Sample for Measuring the Density (ρ)

Each of the solid α-SF-Na products obtained was subjected to screening using sieves having an opening of 16 mm and 150 μm. A fraction passing through a sieve of 16 mm, but not passing through a sieve of 500 μm was used as a sample for measuring the density (ρ).

### (1)-3: Method for Measuring the Density

15 g of the samples for measuring the densities (ρ₀) and (ρ) was separately weighed and the density was measured using a commercially available air pycnometer Model 1000, made by Tokyoscience. Co., Ltd. (measuring conditions: 1-2 atmospheric pressure, measuring atmosphere: RT). The bubble volume fraction was calculated in accordance with the formula (1) as previously mentioned. In light of the errors, the measurement was repeated 10 times for each sample to obtain the average, which was regarded as the bubble volume fraction.

### (2) Method for Determining the Adhesion Properties

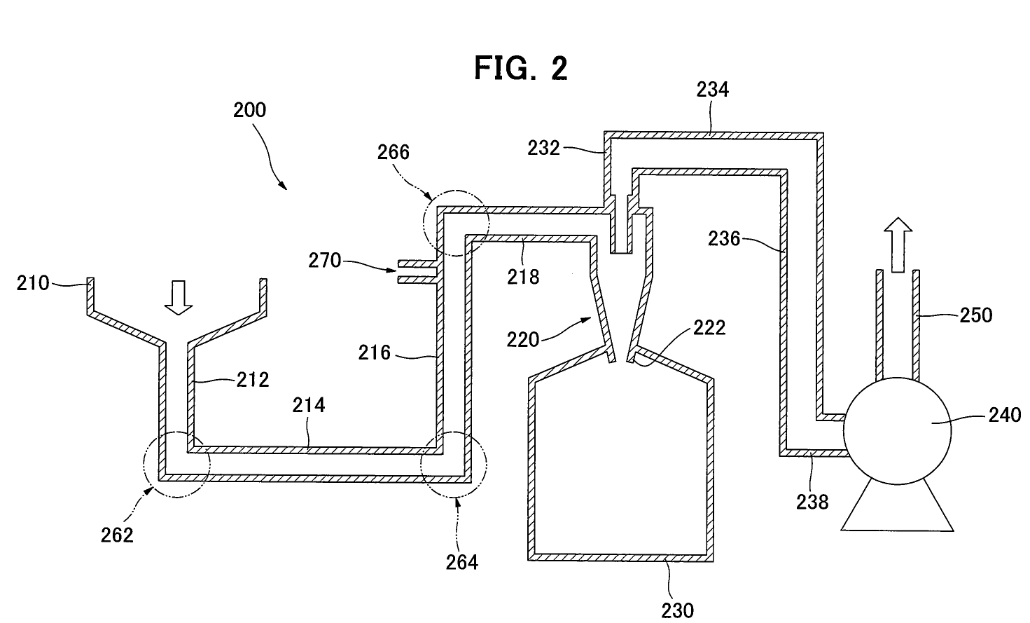

FIG. 2 is a schematic diagram showing a simple acrylic pneumatic conveying system 200 used in the examples of the invention. With reference to FIG. 2, the simple pneumatic conveying system 200 is provided with a sample feed hopper 210 disposed upstream at the upper part, a vertical upstream pipe 212 connected to the sample feed hopper 210 in a downstream direction, a horizontal middle pipe 214 connected to the downstream end of the vertical inlet pipe 212, a vertical middle pipe 216 connected to the downstream end of the horizontal middle pipe 214, a horizontal downstream pipe 218 connected to the downstream end of the vertical middle pipe 216, and a cyclone 220 connected to the horizontal downstream pipe 218 in a downstream direction. A sampling bottle 230 is disposed at the outlet 222 of the cyclone 220. In addition, the simple pneumatic conveying system 200 is provided with a first vertical conveying pipe 232 connected to the cyclone 220, a first horizontal conveying pipe 234 connected to the downstream end of the first vertical conveying pipe 232, a second vertical conveying pipe 236 connected to the downstream end of the first horizontal conveying pipe 234, and a second horizontal conveying pipe 238 connected to the downstream end of the second vertical conveying pipe 236. A suction apparatus 240 is connected to the downstream end of the second horizontal conveying pipe 238. A vertical outlet conveying pipe 250 is connected to the downstream end of the suction apparatus 240. The system of this embodiment is designed so that the length of the vertical inlet pipe 212 is 200 mm; that of the horizontal middle pipe 214, 400 mm; that of the vertical middle pipe 216, 400 mm; and that of the horizontal downstream pipe 218, 305 mm. Further, the system of this embodiment is designed so that the cyclone 220 has an inner diameter of 70 mm and a length of 200 mm. A first elbow 262 for checking the attached material is positioned at a connecting section of the vertical inlet pipe 212 and the horizontal middle pipe 214. A second elbow 264 for checking the attached material is positioned at a connecting section of the horizontal middle pipe 214 and the vertical middle pipe 216. A third elbow 266 for checking the attached material is positioned at a connecting section of the vertical middle pipe 216 and the horizontal downstream pipe 218. A portion 270 for measuring the wind velocity and the wind temperature is located along the vertical middle pipe 216 at a position closer to the third elbow portion 266. Upon operating the suction apparatus 240, wind is generated in the above-mentioned pipes.

In the simple pneumatic conveying system 200, a measuring apparatus (not shown) was placed at the portion 270 for measuring the wind velocity and the wind temperature to previously adjust the wind velocity in the pipes to 20 m/s. Then, a sample was introduced from the sample feed hopper 210 at a feed rate of 0.5 kg/min for two minutes to conduct a conveying test. The amount of the sample attached to each of the three elbows, that is, the first elbow for checking the attached material 262, the second elbow for checking the attached material 264 and the third elbow for checking the attached material 266 was measured, and the evaluation was made in accordance with the following criteria. Prior to the test, the temperature of the wind was 38° C. In light of the errors, the measurement was repeated 10 times for each sample to obtain the average, which was regarded as the adhering properties.

<Evaluation criteria>

| Amount of sample attached | Score |
|---|---|
| Less than 0.5 g: | 1 |
| 0.5 g or more and less than 1 g: | 2 |
| 1 g or more and less than 2 g: | 3 |
| 2 g or more and less than 3 g: | 4 |
| 3 g or more: | 5 |

### (3) Method for Determining the Whiteness Degree

### (3)-1: Pretreatment of Sample

The solid product in the form of granules was sieved using screens of 1000 μm and 500 μm. A sieve fraction having passed through the screen of 1000 μm, but not passing through the screen of 500 μm was used as a sample fraction for measurement.

The solid product in the form of flakes or pellets was subjected to the measurement as it was.

The sample of Example 27 was prepared by cutting a pellet (with a pellet diameter of 10 mm) into a cylindrical form having a bottom with a diameter of 10 mm and a height of 10 mm with a cutter, and the thus prepared sample was subjected to measurement, with the bottom of the cylindrical form being put over the sample plate.

### (3)-2: Method for Determining Hunter Whiteness

The L, a and b values of each sample were measured using a spectrophotometer (SE-2000, made by Nippon Denshoku Industries Co., Ltd.) For the sample in the form of a flake, a sample plate with a diameter of 10 mm was employed and a flake (which was large enough to cover the entire surface of the sample plate) was chosen and set on the sample plate. With a standard white plate being overlaid, the measurement was conducted. For the sample in the form of pellets or granules, the sample was placed into a round-shaped measurement cell designed for powders and subjected to the measurement. From the obtained L, a and b values, the Hunter whiteness degree W (Lab) was calculated in accordance with the following formula:

W(Lab)=100−((100−L)²+a²+b²)^{0.5}

### (3)-3: Measurement of the Value b*

Using a spectrophotometer (SE-2000, made by Nippon Denshoku Industries Co., Ltd.), the value b* (CIE saturation) was measured as the yellowness index. The measuring conditions of the sample were the same as those in the case of the Hunter whiteness degree mentioned in the above item (3)-2. The larger the value b*, the color was evaluated as more yellowish. When the color difference is 1 or more, the difference can be recognized with the naked eye.

In light of the errors, the measurement was repeated 10 times for each sample to obtain the average, which was regarded as the whiteness degree.

### (4) Measurement of the Dimensions of Solid Product

Based on the description in the previously mentioned item [Dimensions of solid α-SF salt product], the shorter diameter and the longer diameter of a solid product in the form of flakes were measured using CAMSIZER made by HORIBA, Ltd. The thickness was measured with vernier calipers.

The diameter and the length of a solid product in the form of pellets were measured with vernier calipers. When the section of the pellet was a circle, the portion corresponding to the diameter of the circular cylinder forming the pellet was regarded as the diameter. When the section was an oval, the average was taken from the longer diameter and the shorter diameter. When the section was a triangle or square, the height from each base of the triangular or square section was measured and the average taken from the heights was determined as the diameter. The portion corresponding to the height of the circular cylinder (or triangular prism or quadrangular prism) constituting the pellet was regarded as the length. The average was taken from 100 solid products in each case.

### Example 29

### Production of Detergent Composition Containing Solid α-SF Salt Product

The raw materials used to produce a detergent compositions are as follows:

Hydrogen peroxide solution: aqueous solution containing 35 mass % of hydrogen peroxide. Extra pure reagent, made by Junsei Chemical Co., Ltd.

Carbonate (Na): granular ash (made by Soda Ash Japan Co., Ltd.)

Fluorescent agent: TINOPAL CBS-X (made by Ciba Specialty Chemicals)

Hydroxide (Na): caustic soda in the form of flakes (made by Tsurumi Soda Co., Ltd.)

Hydroxide (K): caustic potash (made by Asahi Glass Co., Ltd.)

LAS-H: straight-chain alkylbenzenesulfonic acid (LIPON LH-200, made by Lion Corporation), having an AV value (the amount (mg) of potassium hydroxide required to neutralize one gram of LAS-H) of 180.0.

Lauric acid: NAA-122 (made by NOF Corporation.)

STPP: sodium tripolyphosphate (made by Taiyo Kagaku Kogyo)

Silicate (Na): S50° sodium silicate No. 1 (made by Nippon Chemical Industrial Co., Ltd.) with a molar ratio of SiO₂ to Na₂O of 2.15

Polyacrylate (Na): AQUALIC DL-453 (made by Nippon Shokubai Co., Ltd.) (in the form of an aqueous solution substantially containing 35% by mass)

Acrylic acid—maleic acid copolymer (Na): AQUALIC TL-400 (made by Nippon Shokubai Co., Ltd.) (in the form of an aqueous solution substantially containing 40% by mass)

Nonionic surfactant: DIADOL 13 (made by Mitsubishi Chemical Corporation), an adduct of 15 moles (average) of ethylene oxide

Zeolite: Type-A zeolite (with a purity of 47.5% by mass) (made by Nippon Chemical Industrial Co., Ltd.)

Carbonate (K): potassium carbonate (powders) (made by Asahi Glass Co., Ltd.)

Sulfite (Na): sodium sulfite, anhydrous (made by Kamisu Chemical Co., Ltd.)

Sulfate (Na): neutral salt cake, anhydrous (grade: A0) (made by Shikoku Chemicals Corporation)

Soap: sodium salt of fatty acid having 12 to 18 carbon atoms (with a purity of 67% by mass, a titer of 40 to 45° C., and a molecular weight of 289)

Enzyme powder: SAVINASE 18T (made by Novozymes Japan)

Coloring material: 35% solution of blue coloring material (ultramarine blue) (made by Dainichiseika Color & Chemicals Mfg. Co., Ltd.)

Perfume: perfume composition containing: 0.5% of decanal, 0.3% of octanal, 10.0% of hexylcinnamyl aldehyde, 8.0% of dimethyl benzyl carbinyl acetate, 3.0% of lemon oil, 6.0% of lilial, 2.0% of lyral, 5.0% of linalool, 7.5% of phenylethyl alcohol, 2.0% of tonalid, 3.0% of o-tert-butyl cyclohexyl acetate, 2.0% of galaxolide benzyl benzoate, 2.5% of linascol, 1.0% of geraniol, 2.0% of citronellol, 2.0% of jasmorange, 5.0% of methyl dihydro jasmonate, 1.0% of terpineol, 3.0% of methyl ionone, 5.0% of acetyl cedrene, 1.0% of lemonitrile, 1.0% of fruitate, 1.5% of orivone, 1.0% of benzoin, 0.5% of cis-3-hexenol, 2.0% of coumarin, 0.2% of damascenone, 0.3% of damascone, 1.5% of helional, 1.5% of heliotropine, 2.5% of anisaldehyde, 0.8% of gamma undecalactone, 1.2% of bacdanol, 0.5% of triplal, 1.5% of styrallyl acetate, 0.1% of calone, 3.0% of pentalide, 2.9% of oxahexadecen-2-on, and 6.2% of ethylene brassylate (provided that the term “%” of each ingredient indicates the percentage in the perfume composition).

### (Step for Preparation of Slurry)

Water of 25° C. was placed into a blender with an effective capacity of 700 L, provided with a two-stage 45°-pitched paddle type impeller (with an impeller length of 640 mm and an impeller width of 65 mm) and two baffles (having a length of 600 mm and a width of 50 mm, with a clearance of 30 mm between the baffle and the wall surface). Sodium hydroxide was added to water and dissolved therein while the impeller was rotated at 120 rpm. (The stirring operation was continued until the addition of the components was completed). Then, LAS-H was added for neutralization, thereby generating LAS-Na (“LAS-Na” in Table 7 indicates the amount thereof generated after neutralization by the reaction of LAS-H with sodium hydroxide. Generated LAS-Na:sodium hydroxide added:LAS-H added=10.00:1.25:9.36 (ratio by mass)).

Subsequently, builders, i.e., sodium silicate, poly(sodium acrylate), sodium sulfate, sodium tripolyphosphate (STPP), and sodium carbonate were added in this order. With stirring the mixture, steam of 0.1 MPa (in terms of gauge pressure) or cold water of 8° C. was allowed to pass through the jacket of the blender to obtain a detergent slurry of 75° C.

### (Spray-Drying Step)

Then, the detergent slurry was fed to a counter-flow-type dryer tower with a tower diameter of 2.0 m and an effective length of 5.6 m from the top thereof at a feed rate of 400 kg/h., and sprayed using a pressure nozzle, so that spray-dried granules having a water content of 5% were obtained. The spraying operation was carried out under a spraying pressure of 2 to 3.5 MPa using the same nozzle as that described in Example 2 of JP Kokai No. Hei 9-75786. The temperature of heated air in the drying tower was controlled within a range of 270 to 400° C. so that the spray-dried granules might have a water content of 5%. The exhaust rate was 240 m³/min.

### (Mixing Step)

The thus prepared spray-dried granules and the solid α-SF-Na product obtained in Example 8, 11, 26 or 27 were placed into a horizontal drum-shaped rolling mixer (with a drum diameter of 585 mm, a drum length of 490 mm and a container of 131.7 L, with two 45-mm-high baffles being installed with a clearance of 20 mm between the baffle and the inner wall surface of the drum) at a loading ratio of 30%. While the spray-dried granules were mixed at 22 rpm and 25° C., the nonionic surfactant and the perfume in such amounts as indicated in Table 7 were sprayed onto the spray-dried granules to impart a fragrance thereto, so that a granular detergent composition was obtained.

### Example 30

### Step for Preparation of Slurry

Water of 25° C. was placed into a blender with an effective capacity of 700 L, provided with a two-stage 45°-pitched paddle type impeller (with an impeller length of 640 mm and an impeller width of 65 mm) and two baffles (having a length of 600 mm and a width of 50 mm, with a clearance of 30 mm between the baffle and the wall surface). A fluorescent agent and potassium hydroxide were added to water and dissolved therein while the impeller was rotated at 120 rpm. (The stirring operation was continued until the addition of the components was completed). Then, LAS-H was added for neutralization, thereby generating LAS-K (“LAS-K” in Table 7 indicates the amount thereof generated after neutralization by the reaction of LAS-H with potassium hydroxide. Generated LAS-K:potassium hydroxide added:LAS-H added=10.00:1.67:8.94 (ratio by mass)).

Subsequently, sodium hydroxide was added and lauric acid previously melted by adjusting to 60° C. was added for neutralization, thereby generating sodium laurate (“Laurate (Na)” in Table 6 indicates the amount thereof generated after neutralization by the reaction of lauric acid with sodium hydroxide. Generated laurate (Na):sodium hydroxide added:lauric acid added=10.0:2.0:11.1 (ratio by mass)).

Then, builders, i.e., copolymer sodium salt of acrylic acid and maleic acid, zeolite (not including the amount corresponding to 5.0% to be used as the grinding aid and 1.5% as the surface modifier), potassium carbonate, sodium sulfate, sodium sulfite, and sodium carbonate were added in this order.

With stirring the mixture, steam of 0.1 MPa (in terms of gauge pressure) or cold water of 8° C. was allowed to pass through the jacket of the blender to obtain a detergent slurry of 75° C.

### (Spray-Drying Step)

Then, the detergent slurry was subjected to spray-drying operation in the same manner as in Example 29. The water content of the obtained spray-dried granules was 7.5%.

### (Kneading and Extrusion Step)

The obtained spray-dried granules, the nonionic surfactant (not including the amount corresponding to 0.5% to be used for surface modification) and the solid α-SF-Na product obtained in Example 27 were continuously fed into a feed hopper of a continuous kneader (KRC kneader, model S-4, made by Kurimoto, Ltd.) at a feed rate of 225 kg/h and kneaded under the conditions that the number of revolutions of the main shaft was 130 rpm and the kneading energy was 30 kJ/kg. Then, the kneaded product of 55° C. was obtained.

The kneaded product thus obtained was then continuously fed into an extruder (Pelleter Double EXDJF-100, made by Dalton Co., Ltd., having a die with an orifice diameter of 10 mm) at a feed rate of 225 kg/h. Upon extrusion, the extruded product was cut with a cutter (having a peripheral speed of 5 m/s), so that a solid detergent in the form of pellets having a length of about 5 to 30 mm was obtained. The extrusion conditions were that the number of revolutions of the main shaft was 72 rpm, the kneading energy was 10 kJ/kg, and the jacket temperature was 25° C. The temperature of the thus extruded product was 60° C.

### (Crushing Step)

Then, particles of the A type zeolite (with an average particle size of 180 μm) were added to the solid detergent composition in an amount of 5.0% as the grinding aid, followed by crushing in a stream of cold air (10° C., 15 m/s) using a pulverizer, Fitzmill DKA-3 made by Hosokawa Micron Corporation where three Fitzmills were arranged in series (the first, second and third Fitzmill's screens having the respective opening diameters of 6 mm, 4 mm and 2 mm, and the number of revolutions of 4700 rpm in any stage).

### (Post-Treatment Step)

### (1) Surface Modification

The crushed product thus obtained was placed into a horizontal drum-shaped rolling mixer (with a drum diameter of 585 mm, a drum length of 490 mm and a container of 131.7 L, with two 45-mm-high baffles being installed with a clearance of 20 mm between the baffle and the inner wall surface of the drum). Finely-divided particles of the A type zeolite were added to the crushed product in an amount of 1.5%, was mixed together for one minute under the conditions that the loading ratio was 30 vol. %, the number of revolutions was 22 rpm and the temperature was 25° C., with the nonionic surfactant in an amount of 0.5% and the perfume being sprayed onto the contents in the mixer to achieve the surface modification. Then, detergent granules were obtained.

### (2) Coloring of Detergent Granules

After imparting a fragrance to the detergent granules, part of the detergent granules were colored in such a manner that a solution of blue coloring material was sprayed onto the surface of the detergent granules while the detergent granules were carried on a belt conveyer at a speed of 0.5 m/s (with the detergent granules forming a layer with a height of 30 mm and a width of 300 mm on the belt conveyor). Then, detergent granules (with an average particle size of 500 μm and a bulk density of 0.89 g/mL) were obtained.

(3) Mixing with Other Particles

After coloring the detergent granules, the enzyme powder in an amount of 1.0% was mixed with the detergent granules in a horizontal drum-shaped rolling mixer (with a drum diameter of 585 mm, a drum length of 490 mm and a container of 131.7 L, with two 45-mm-high baffles being installed with a clearance of 20 mm between the baffle and the inner wall surface of the drum) for 5 minutes under the conditions that the loading ratio was 30 vol. %, the number of revolutions was 22 rpm and the temperature was 25° C. Then, detergent composition (with an average particle size of 500 μm and a bulk density of 0.89 g/mL) were obtained.

TABLE 1

| | | | | | | Comp. | Comp. |
|---|---|---|---|---|---|---|---|
| | | Ex. 1 | Ex. 2 | Ex. 3 | Ex. 4 | Ex. 1 | Ex. 2 |
| Introduction of | | ◯ | ◯ | ◯ | ◯ | — | — |
| compressed air | | | | | | | |
| Addition of expanding | | — | — | — | — | — | — |
| agent | | | | | | | |
| Temp. (° C.) of paste | | 110 | 110 | 110 | 110 | — | — |
| fed into kneader | | | | | | | |
| Feed rate (kg/h) of | | 90 | 50 | 20 | 10 | — | — |
| paste into kneader | | | | | | | |
| Kneading energy | | 1.0 | 1.8 | 4.5 | 9.0 | — | — |
| (kJ/kg) | | | | | | | |
| Temp. (° C.) of | | 90 | 80 | 70 | 65 | — | — |
| kneaded product | | | | | | | |
| Bubble volume | | 1 | 1 | 3 | 5 | 0 | 0 |
| fraction (%) | | | | | | | |
| Adhesion properties | | 3 | 3 | 1 | 1 | 5 | 5 |
| (score) | | | | | | | |
| Whiteness | W (Lab) | 70 | 70 | 74 | 80 | 66 | 55 |
| degree | b* value | 19 | 18 | 13 | 12 | 21 | 25 |
| Solid | Shape | flakes | flakes | flakes | flakes | flakes | flakes |
| product | Thickness | 2 | 2 | 2 | 2 | 2 | 6 |
| | (mm) | | | | | | |
| | Longer | 20 | 18 | 23 | 21 | 21 | 22 |
| | diameter | | | | | | |
| | (mm) | | | | | | |
| | Shorter | 10 | 13 | 14 | 9 | 10 | 12 |
| | diameter | | | | | | |
| | (mm) | | | | | | |

TABLE 2

| | | | | | | Comp. |
|---|---|---|---|---|---|---|
| | | Ex. 5 | Ex. 6 | Ex. 7 | Ex. 8 | Ex. 3 |
| Introduction of | | ◯ | — | — | ◯ | — |
| compressed air | | | | | | |
| Addition of expanding | | — | — | ◯ | — | — |
| agent | | | | | | |
| Temp. (° C.) of paste | | 110 | 110 | 110 | 110 | — |
| fed into kneader | | | | | | |
| Feed rate (kg/h) of | | 20 | 20 | — | 90 | — |
| paste into kneader | | | | | | |
| Kneading energy | | 4.5 | 4.5 | 2.0 | 1.0 | — |
| (kJ/kg) | | | | | | |
| Temp. (° C.) of | | 70 | 70 | 70 | 90 | — |
| kneaded product | | | | | | |
| Bubble volume | | 11 | 3 | 14 | 1 | 0 |
| fraction (%) | | | | | | |
| Adhesion properties | | 1 | 1 | 3 | 3 | 5 |
| (score) | | | | | | |
| Whiteness | W (Lab) | 85 | 72 | 82 | 90 | 78 |
| degree | b* value | 10 | 14 | 14 | 8 | 16 |
| Solid | Shape | flakes | flakes | flakes | granules | granules |
| product | Thickness (mm) | 6 | 2 | 6 | — | — |
| | Longer diameter | 30 | 21 | 34 | — | — |
| | (mm) | | | | | |
| | Shorter diameter | 22 | 13 | 27 | — | — |
| | (mm) | | | | | |

TABLE 3

| | | Ex. 9 | Ex. 10 | Ex. 11 | Ex. 12 | Ex. 13 | Ex. 14 |
|---|---|---|---|---|---|---|---|
| Introduction of | | Not introduced | | | | | |
| compressed air | | (but the feed hopper was left open) | | | | | |
| Addition of expanding | | Not added | | | | | |
| agent | | | | | | | |
| Third orifice plate of | | circle | circle | circle | circle | circle | circle |
| extruder: Shape of | | | | | | | |
| orifice | | | | | | | |
| Orifice diameter (mm) | | 0.4 | 0.5 | 0.8 | 1.0 | 1.2 | 1.5 |
| Area of orifice | | 0.13 | 0.20 | 0.50 | 0.79 | 1.13 | 1.77 |
| (mm²/orifice) | | | | | | | |
| Feed rate (kg/h) of | | 20 | 50 | 80 | 80 | 80 | 80 |
| flakes into kneader | | | | | | | |
| Kneading energy | | 140 | 75 | 41 | 38 | 33 | 30 |
| (kJ/kg) | | | | | | | |
| Temp. (° C.) of kneaded | | 75 | 70 | 65 | 65 | 63 | 65 |
| product | | | | | | | |
| Bubble volume | | 15 | 14 | 8 | 7 | 6 | 4 |
| fraction (%) | | | | | | | |
| Adhesion properties | | 3 | 3 | 1 | 1 | 1 | 1 |
| (score) | | | | | | | |
| Whiteness degree | W (Lab) | 87 | 87 | 83 | 82 | 80 | 78 |
| | b* value | 10 | 10 | 14 | 14 | 15 | 16 |
| Solid product | Shape | pellets | pellets | pellets | pellets | pellets | pellets |
| | Diameter (mm) | 0.4 | 0.5 | 0.8 | 1.0 | 1.2 | 1.5 |
| | Length | 1.8 | 2.0 | 2.5 | 2.8 | 3.0 | 4.5 |

TABLE 4

| | | Ex. 15 | Ex. 16 | Ex. 17 | Ex. 18 | Ex. 19 | Ex. 20 |
|---|---|---|---|---|---|---|---|
| Introduction of | | Not introduced | | | | | |
| compressed air | | (but the feed hopper was left open) | | | | | |
| Addition of expanding | | Not added | | | | | |
| agent | | | | | | | |
| Third orifice plate of | | circle | circle | circle | circle | circle | circle |
| extruder: Shape of | | | | | | | |
| orifice | | | | | | | |
| Orifice diameter (mm) | | 1.9 | 0.8 | 0.8 | 0.8 | 0.8 | 0.8 |
| Area of orifice | | 2.83 | 0.50 | 0.50 | 0.50 | 0.50 | 0.50 |
| (Extruding area) | | | | | | | |
| (mm²/orifice) | | | | | | | |
| Feed rate (kg/h) of | | 80 | 40 | 60 | 70 | 100 | 120 |
| flakes into kneader | | | | | | | |
| Kneading energy | | 27 | 30 | 34 | 38 | 47 | 62 |
| (kJ/kg) | | | | | | | |
| Temp. (° C.) of kneaded | | 65 | 50 | 55 | 60 | 70 | 75 |
| product | | | | | | | |
| Bubble volume | | 3 | 14 | 12 | 10 | 6 | 5 |
| fraction (%) | | | | | | | |
| Adhesion properties | | 1 | 3 | 2 | 1 | 1 | 1 |
| (score) | | | | | | | |
| Whiteness degree | W (Lab) | 76 | 87 | 85 | 84 | 82 | 81 |
| | b* value | 17 | 10 | 12 | 13 | 14 | 15 |
| Solid product | Shape | pellets | pellets | pellets | pellets | pellets | pellets |
| | Diameter (mm) | 1.9 | 0.8 | 0.8 | 0.8 | 0.8 | 0.8 |
| | Length (mm) | 5.2 | 2.2 | 2.3 | 2.5 | 2.4 | 2.4 |

TABLE 5

| | | Ex. 21 | Ex. 22 | Ex. 23 | Ex. 24 |
|---|---|---|---|---|---|
| Introduction of | | Not introduced | | | |
| compressed air | | (but the feed hopper was left open) | | | |
| Addition of expanding | | Not added | | | |
| agent | | | | | |
| Third orifice plate of | | circle | circle | regular | regular |
| extruder: Shape of | | | | triangle with | square with |
| orifice Orifice | | | | a side of | a side of |
| diameter (mm) | | | | 0.75 mm | 0.7 mm |
| Area of orifice | | 0.8 | 0.8 | — | — |
| (Extruding area) | | 0.50 | 0.50 | 0.49 | 0.49 |
| (mm²/orifice) | | | | | |
| Feed rate (kg/h) of | | 150 | 200 | 80 | 80 |
| flakes into kneader | | | | | |
| Kneading energy | | 69 | 71 | 41 | 41 |
| (kJ/kg) | | | | | |
| Temp. (° C.) of kneaded | | 80 | 85 | 65 | 65 |
| product | | | | | |
| Bubble volume | | 4 | 3 | 8 | 8 |
| fraction (%) | | | | | |
| Adhesion properties | | 1 | 1 | 1 | 1 |
| (score) | | | | | |
| Whiteness | W (Lab) | 78 | 76 | 87 | 85 |
| degree | b* value | 16 | 17 | 10 | 12 |
| Solid | Shape | pellets | pellets | pellets | pellets |
| product | Diameter | 0.8 | 0.8 | 0.75/side | 0.7/side |
| | (mm) | | | | |
| | Length | 2.6 | 2.5 | 2.7 | 2.5 |
| | (mm) | | | | |

TABLE 6

| | | Ex. 25 | Ex. 26 | Ex. 27 | Ex. 28 |
|---|---|---|---|---|---|
| Introduction of compressed air | | Not introduced | | | |
| | | (but the feed hopper was left open) | | | |
| Addition of expanding agent | | Not added | | | |
| Kneading | Feed rate (kg/h) of | 225 | 225 | 225 | 225 |
| | paste into kneader | | | | |
| | Kneading energy | 30 | 11 | 11 | 11 |
| | (kJ/kg) | | | | |
| | Temp. (° C.) of | 55 | 52 | 52 | 52 |
| | kneaded product | | | | |
| Extrusion | Feed rate (kg/h) of | 55 | 52 | 52 | 52 |
| | kneaded product into | | | | |
| | extruder | | | | |
| | Shape of orifice | circle | circle | circle | circle |
| | Orifice diameter (mm) | 0.8 | 0.8 | 5 | 10 |
| | Area of orifice | 0.50 | 0.50 | 19.63 | 78.54 |
| | (Extruding area) | | | | |
| | (mm²/orifice) | | | | |
| | Kneading energy | 10 | 9 | 7 | 7 |
| | (kJ/kg) | | | | |
| | Temp. (° C.) of | 60 | 55 | 55 | 55 |
| | extruded product | | | | |
| Cooling step | | ◯ | ◯ | ◯ | ◯ |
| Crushing step | | ◯ | ◯ | ◯ | ◯ |
| Pulverizing step | | ◯ | ◯ | ◯ | — |
| Bubble volume fraction (%) | | 10 | 12 | 10 | 12 |
| Adhesion properties (score) | | 1 | 1 | 1 | 1 |
| Whiteness | W (Lab) | 87 | 85 | 91 | 82 |
| degree | b* value | 10 | 12 | 7 | 14 |
| Solid | Shape | pellets | pellets | granules | pellets |
| product | Diameter (mm) | 0.8 | 0.8 | — | 10 |
| | Length (mm) | 2.6 | 2.5 | — | 18 |

TABLE 7

| | | Example 29 | Example 30 |
|---|---|---|---|
| Composition | α-SF-Na | 10 | 10 |
| of | LAS-Na | 10 | 0 |
| Detergent | LAS-K | 0 | 2 |
| Granules | Laurate (Na) | 0 | 5 |
| (%) | Nonionic surfactant | 2 | 4 |
| | STPP | 20 | 0 |
| | Silicate (Na) | 10 | 0 |
| | Polyacrylate (Na) | 2 | 0 |
| | Acrylic acid - maleic acid | 0 | 2 |
| | copolymer (Na) | | |
| | Sulfate (Na) | 24 | 14 |
| | Carbonate (Na) | 15 | 28 |
| | Carbonate (K) | 0 | 5 |
| | Sulfite (Na) | 0 | 2 |
| | Zeolite | 0.4 | 15 |
| | Fluorescent agent | 0 | 0.1 |
| | Enzyme | 0 | 1 |
| | Perfume | 0.2 | 0.2 |
| | Coloring material | 0 | 0.015 |
| | Other minor components | balance | balance |
| | Total | 100 | 100 |

### REFERENCE SIGNS LIST

100 Screw extrusion granulator
110 Casing
112 Feed hopper
114 Binder feed opening
118 Product outlet
122 First orifice plate
124 Second orifice plate
126 Third orifice plate
130 Latch
140 Screw shaft
144 First paddle
146 Second paddle
150 Jacket
200 Simple pneumatic conveying system
210 Sample feed hopper
212 Vertical upstream pipe
214 Horizontal middle pipe
216 Vertical middle pipe
218 Horizontal downstream pipe
220 Cyclone
230 Sampling bottle
232 First vertical conveying pipe
234 First horizontal conveying pipe
236 Second vertical conveying pipe
238 Second horizontal conveying pipe
240 Suction apparatus
250 Vertical outlet conveying pipe
262 First elbow for checking the attached material
264 Second elbow for checking the attached material
266 Third elbow for checking the attached material
270 Portion for measuring wind velocity and wind temperature

1. A solid a-sulfo fatty acid alkyl ester salt having a bubble volume fraction of 1 to 15%.

2. The solid a-sulfo fatty acid alkyl ester salt of claim 1, wherein the solid is in the form of pellets having an average diameter of 0.3 to 15 mm and an average length of 0.3 to 100 mm.

3. The solid a-sulfo fatty acid alkyl ester salt of claim 1, wherein the bubbles have an average diameter of 500 μm or less.

4. A detergent composition comprising the solid a-sulfo fatty acid alkyl ester salt of claim 1.

5. A method for producing a solid a-sulfo fatty acid alkyl ester salt having a bubble volume fraction of 1 to 15%, comprising the step of incorporating bubbles into a paste of a-sulfo fatty acid alkyl ester salt.

6. The method of claim 5, wherein the bubbles are incorporated into the paste of a-sulfo fatty acid alkyl ester salt by kneading the paste of a-sulfo fatty acid alkyl ester salt, with a gas being introduced therein.

7. The method of claim 6, wherein the gas is introduced in an amount of 10⁻⁵ to 10⁻² Nm3 per kilogram of the paste of a-sulfo fatty acid alkyl ester salt.

8. The method of claim 6, wherein the paste of a-sulfo fatty acid alkyl ester salt is kneaded by the application of a kneading energy of 0.1 to 50 kJ/kg.

9. The method of claim 5, wherein the bubbles are incorporated into the paste of a-sulfo fatty acid alkyl ester salt by kneading the paste of a-sulfo fatty acid alkyl ester salt in the presence of an expanding agent.

10. The method of claim 6, wherein the paste of a-sulfo fatty acid alkyl ester salt has a temperature of 40 to 95° C. after kneading.

11. A method for producing a solid a-sulfo fatty acid alkyl ester salt having a bubble volume fraction of 1 to 15%, comprising the step of kneading a solid-state a-sulfo fatty acid alkyl ester salt to incorporate bubbles therein, while turning the solid-state a-sulfo fatty acid alkyl ester salt into a paste.

12. The method of claim 11, wherein the solid-state a-sulfo fatty acid alkyl ester salt is kneaded by the application of a kneading energy of 10 to 500 kJ/kg, with a feed hopper of a kneader being left open.

13. The method of claim 5, further comprising the step of passing the kneaded paste of a-sulfo fatty acid alkyl ester salt through an extruder die.

14. The method of claim 13, wherein the extruder die has an extrusion area of 0.1 to 20 200 mm²/orifice.

15. The method of claim 5, wherein the solid product is in the form of pellets with an average diameter of 0.3 to 2 mm and an average length of 0.3 to 10 mm.

16. The method of claim 5, wherein the bubbles contained in the solid a-sulfo fatty acid alkyl ester salt have an average diameter of 500 μm or less.
